(19) United States
(12) Patent Application Publication
(10) Pub. No.: US 2024/0058620 A1
(43) Pub. Date: Feb. 22, 2024

(54) SYSTEM AND METHOD OF LASER THERAPY APPLIED AT DURATIONS IN RANGE OF 1US TO 9US

(71) Applicant: Bruce Coren, West Palm Beach, FL (US)

(72) Inventors: Bruce Coren, West Palm Beach, FL (US); Joseph Costello, West Palm Beach, FL (US)

(21) Appl. No.: 17/890,919

(22) Filed: Aug. 18, 2022

(51) Int. Cl. A61N 5/06; A61N 5/067

(52) U.S. Cl. CPC A61N 5/0613; A61N 5/067; A61N 2005/0626; A61N 2005/0642

(57) ABSTRACT

A method for treating pain using laser therapy in pulse mode is provided. The method comprises a laser receiving a power setting in Watts, the laser receiving a setting of cycles in Hz, and the laser administering a laser treatment of 810 nm wavelength of duration T-on between 1 us and 9us. The method also comprises the laser calculating a T-off, a time off between pulses of the laser treatment and the laser implementing the T-off. Selections of duration between 1 us-9us are dependent at least on selection of power (Watts) (100W-250W). Selected durations between 1us-9us are directed to reducing thermal effect on a patient subjected to the laser treatments. Selection of duration between 1 us-9us facilitates steady or static placement of a laser providing the treatment dosage over an area of a pathology.

### CROSS REFERENCE TO RELATED APPLICATIONS

None

### FIELD OF THE INVENTION

The present disclosure is in the laser field of physical medicine and therapy. More particularly, the present disclosure provides systems and methods for the treatment of pain utilizing a high-power class IV medical laser with treatment applied to affected areas with durations in range of 1 us to 9 us.

### BACKGROUND

Lasers used in the physical medicine and therapy field are an effective analgesic for treating pain for numerous ailments. Such treatments are limited in their effectiveness when treating deeply located areas of pain in the body, due at least to the absorption coefficient of wavelengths into the mammalian body mass.

Laser treatments for pain are typically applied using one of two energy output modes. One is to run the laser light continuously or in continuous wave (CW) mode. The second is to pulse the laser light in pulse mode (PM), which effectively pauses the laser emissions in given beats and rhythms.

### BRIEF DESCRIPTION OF THE FIGURES

FIG. 1 is a block diagram of a system of laser therapy applied at durations in range of 1 us to 9 us according to an embodiment of the present disclosure.

FIG. 2 is a graph illustrating relative absorption of water and hemoglobin at various wavelengths according to an embodiment of the present disclosure.

### DETAILED DESCRIPTION

Systems and methods described herein provide for applying photobiomodulation laser treatment to affected areas of neuromusculoskeltal pain for durations of 1 us to 9 us. Treatment at those durations may eliminate heat and allow for steady positioning of laser device over affected areas without movement. The range of 1 us to 9 us provides a means to use high power class IV laser of 100 W to 250 W to remove or materially change an amount of thermal input to a target tissue or pathology. When a pathology is small and focal, thermal input may be effectively removed. The range of 1 us to 9 us is of interest because based on a power setting of 100 W to 250 W, it may be necessary to deliver a static focal treatment without thermal buildup. For example, at 250 W and 5 Hz at a Ton of 2 us, there is no thermal buildup affecting superficial tissues.

A setting of 1-9 us carries sufficient energy to heal photobiomodulation but is fast enough to eliminate heat. Healing effect is stimulated by energy. The width of the pulse is sufficient to deliver an effective dosage of laser but narrow enough not to be detected by tissues.

In an example, a laser receives a power setting and administers a treatment of 810 nm wavelength of duration between 1 us-9 us. The treatment is followed by a pause determined by systems and methods provided herein.

Selections of duration between 1 us-9 us are dependent upon the level of joules to stimulate healing effect while limiting thermal effect. A Toff quench sequence provides cellular membrane relaxation and ionic transfer. Steady placement over an area of treatment provides effective treatment based on an attenuated thermal effect.

Selections of duration between 1 us-9 us may be directed to patients afflicted with spinal stenosis. The selections between 1 us-9 us are directed to chronic thickening of ligament flavum and relief of spinal stenosis. The selections are not affected by presence or absorption of water, hemoglobin, or melatonin.

In another example, a high-powered laser receives activation to deliver a static focal treatment and receives an initial power setting of 100 W-250 W. Based on a determined need to deliver the treatment without thermal buildup, Ton is selected in a range of 1 us-9 us.

In the example, the selection of duration is directed to achieving level of joules to stimulate healing effect while limiting thermal effect.

In yet another example, based on a determination that a pathology is small and focal, a high-powered laser receives a power setting of 100 W-250 W for a treatment. The laser receives a T-on duration setting in a range of 1 us-9 us for the treatment. Based on administration of the laser treatment at the above power and duration settings, a quantity of thermal input to a target tissue and the pathology is removed or modified.

The advantage of a stealth MicroPulse laser system described herein is that it can deliver more energy (joules) because it pulses in millions of a second (microseconds) vs superpulsed lasers which pulse in billions of a second(nanoseconds) or even trillions of a second (picoseconds). These extremely short bursts of energy carry far less energy per pulse.

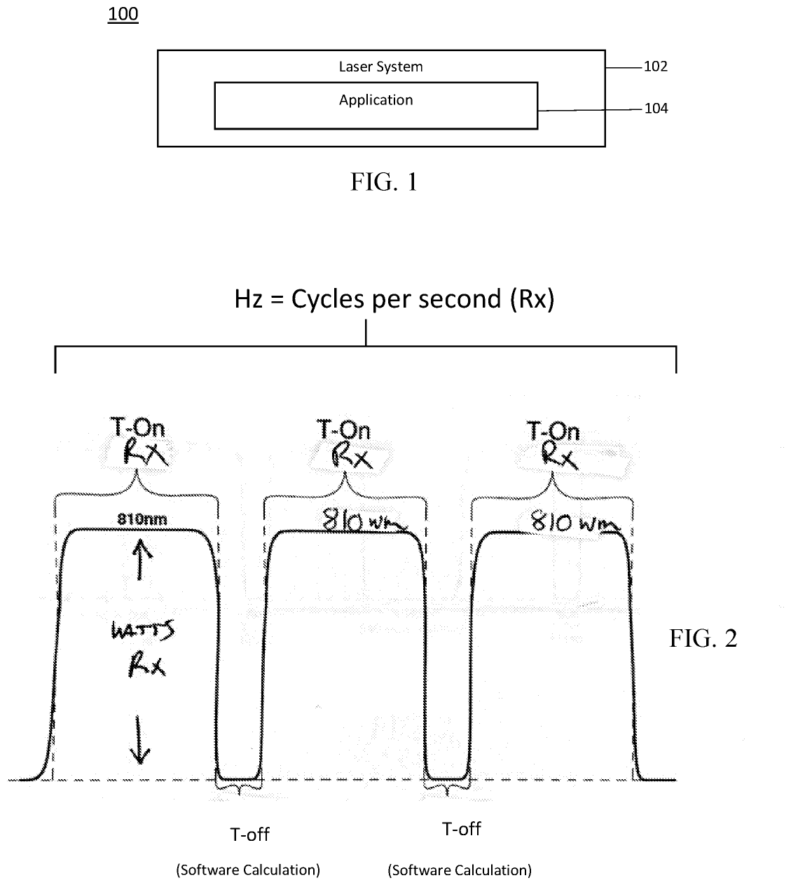

The stealth MicroPulse laser system referred to above and the methods that execute on the system's associated methods comprise a combination of hardware and software. FIG. 1 illustrates components of a system 100 of laser therapy applied at durations in range of 1 us to 9 us according to an embodiment of the present disclosure. The system 100 comprises a laser 102 and an application 104. The laser 102 is a high-power laser with some handheld components. The terms “stealth” and MicroPulse may be commercially applied terms for the system when it is sold and distributed in market settings. The application 104 executes on the laser 104. The application 104 makes calculations based on entries of power setting and setting of cycles in Hz and the administration of the laser treatment of 810 nm wavelength of duration between 1 us and 9 us. The application determines the correct T-off based on these settings and based on other factors. FIG. 2 is a graph illustrating relative absorption of water and hemoglobin at various wavelengths. Absorption of water is particularly high at around 965 nm.

In an embodiment, a method for treating pain using laser therapy in pulse mode is provided. The method comprises a laser receiving a power setting in Watts, the laser receiving a setting of cycles in Hz, and the laser administering a laser treatment of 810 nm wavelength of duration T-on between 1 us and 9 us. The method also comprises the laser calculating a T-off, a time off between pulses of the laser treatment and the laser implementing the T-off.

Selections of duration between 1 us-9 us are dependent at least on selection of power (Watts) (100 W-250 W). Selected durations between 1 us-9 us are directed to reducing thermal effect on a patient subjected to the laser treatments. Selection of duration between 1 us-9 us facilitates steady or static placement of a laser providing the treatment dosage over an area of a pathology.

The steady or static placement over the area of the treatment provides effective treatment based on modulated or attenuated thermal effect. Selection of duration between 1 us-9 us is directed to achieving level of joules to stimulate healing effect while limiting thermal effect. A T-on (us) and a T-off (quench sequence) provide static focal treatment to a desired pathology.

Selections of duration between 1 us-9 us are directed to patients afflicted with spinal stenosis. Selections of duration between 1 us-9 us are directed to chronic thickening of ligament flavum and relief of spinal stenosis. Selections of duration between 1 us-9 us at 810 nm are least affected by presence or absorption of water, hemoglobin, or melatonin.

In another embodiment, a system for treating pain using laser therapy. The system comprises a high-powered laser that receives activation to deliver a static focal treatment and receives an initial power setting of 100 W-250 W at a first T-on setting to deliver safe, non-thermal deep treatment. Based on a determined need to deliver the treatment without thermal buildup, the system adjusts T-off.

Selection of duration between 1 us-9 us is directed to achieving level of joules to stimulate healing effect while limiting thermal effect. A T-off quench sequence provides static focal treatment to a desired pathology. Selections of duration between 1 us-9 us are directed to patients afflicted with spinal stenosis.

In yet another embodiment, a system for applying laser treatments in durations ranging from 1 us to 9 us is provided. The system comprises a high-powered laser that based on a determination that a pathology is small and focal, receives a power setting for a laser treatment of 100 W-250 W (high peak power). The system also receives a T-on duration setting in a range of 1 us-9 us for the laser treatment. Based on the laser treatment at the power and duration settings, the system also one of removes and modifies quantity of thermal input to a target tissue and the pathology.

The treatment promotes placement of the laser over the target tissue without movement. A duration of 1-9 us provides sufficient joules and eliminates heat.

Selections of duration between 1 us-9 us are directed to patients afflicted with spinal stenosis. Selections of duration between 1 us-9 us are directed to chronic thickening of ligament flavum and relief of spinal stenosis. Selections of duration between 1 us-9 us (810 nm) are least affected by presence or absorption of water, hemoglobin, or melatonin.

What is claimed is:

1. A method for treating pain using laser therapy in pulse mode comprising:
a laser receiving a power setting in Watts;
the laser receiving a setting of cycles in Hz;
the laser administering a laser treatment of 810 nm wavelength of duration T-on between 1 us and 9 us;
the laser calculating a T-off, a time off between pulses of the laser treatment; and
the laser implementing the T-off.

2. The method of claim 1, wherein selections of duration between 1 us-9 us are dependent at least on selection of power (Watts) (100 W-250 W).

3. The method of claim 1, wherein selected durations between 1 us-9 us are directed to reducing thermal effect on a patient subjected to the laser treatments.

4. The method of claim 1, wherein selection of duration between 1 us-9 us facilitates steady or static placement of a laser providing the treatment dosage over an area of a pathology.

5. The method of claim 4, wherein the steady or static placement over the area of the treatment provides effective treatment based on modulated or attenuated thermal effect.

6. The method of claim 1, wherein selection of duration between 1 us-9 us is directed to achieving level of joules to stimulate healing effect while limiting thermal effect.

7. The method of claim 1, wherein a T-on (us) and a T-off (quench sequence) provide static focal treatment to a desired pathology.

8. The method of claim 1, wherein selections of duration between 1 us-9 us are directed to patients afflicted with spinal stenosis.

9. The method of claim 1, wherein selections of duration between 1 us-9 us are directed to chronic thickening of ligament flavum and relief of spinal stenosis.

10. The method of claim 1, wherein selections of duration between 1 us-9 us at 810 nm are least affected by presence or absorption of water, hemoglobin, or melatonin.

11. A system for treating pain using laser therapy comprising:
a high-powered laser that:
receives activation to deliver a static focal treatment,
receives an initial power setting of 100 W-250 W at a first T-on setting to deliver safe, non-thermal deep treatment,
based on a determined need to deliver the treatment without thermal buildup, adjusts T-off.

12. The system of claim 11, wherein selection of duration between 1 us-9 us is directed to achieving level of joules to stimulate healing effect while limiting thermal effect.

13. The system of claim 11, wherein a T-off quench sequence provides static focal treatment to a desired pathology.

14. The system of claim 11, wherein selections of duration between 1 us-9 us are directed to patients afflicted with spinal stenosis.

15. A system for applying laser treatments in durations ranging from 1 us to 9 us, comprising:
a high-powered laser that:
based on a determination that a pathology is small and focal, receives a power setting for a laser treatment of 100 W-250 W (high peak power),
receives a T-on duration setting in a range of 1 us-9 us for the laser treatment,
based on the laser treatment at the power and duration settings, one of removes and modifies quantity of thermal input to a target tissue and the pathology.

16. The system of claim 15, wherein the treatment promotes placement of the laser over the target tissue without movement.

17. The system of claim 15, wherein a duration of 1-9 us provides sufficient joules and eliminates heat.

18. The system of claim 15, wherein selections of duration between 1 us-9 us are directed to patients afflicted with spinal stenosis.

19. The system of claim 15, wherein selections of duration between 1 us-9 us are directed to chronic thickening of ligament flavum and relief of spinal stenosis.

20. The system of claim 15, wherein selections of duration between 1 us-9 us (810 nm) are least affected by presence or absorption of water, hemoglobin, or melatonin.
